(19) United States
(12) Patent Application Publication
(10) Pub. No.: US 2011/0182955 A1
(43) Pub. Date: Jul. 28, 2011

(54) WATER- AND ACTIVE INGREDIENT-CONTAINING GEL

(76) Inventors: Michael Roreger, Neuwied (DE); Iris Schnitzler, Bonn (DE); Christian Hausen, Vettelschoss (DE)

(21) Appl. No.: 12/673,046

(22) PCT Filed: Aug. 12, 2008
(86) PCT No.: PCT/EP2008/006620

§ 371 Date: Feb. 11, 2010

(30) Foreign Application Priority Data
Aug. 20, 2007 (DE) 10 2007 039 229.1

(51) Int. Cl. A61K 8/02; A61K 8/81; A61K 8/73; A61Q 19/00; A61Q 19/08

(52) U.S. Cl. 424/401; 514/629

(57) ABSTRACT

The invention relates to a hydrogel formed from water, at least one gel former, at least one pharmaceutical active ingredient or cosmetic ingredient and, optionally, further auxiliaries and additives obtainable by a) mixing water and optionally including auxiliaries and additives when appropriate, b) adding the gel former and homogenizing the mixture, c) adding the pharmaceutical active ingredient or cosmetic ingredient and, optionally, ionic compounds which induce gel formation, and homogenizing the mixture, d) pouring out the mixture onto a support and e) cooling the mixture to form the hydrogel, where steps a) to d) are carried out at a temperature between 79 and 95° C. This hydrogel is suitable for administering cosmetic ingredients onto or via the skin or mucous membrane of a human, in particular in the form of a cosmetic face pack.

The present invention relates to a water-containing gel (hydrogel) which, besides water, comprises at least one gel former and at least one pharmaceutical and/or one cosmetic (active) ingredient. The gel according to the invention preferably comprises at least one cosmetic ingredient. Furthermore, the invention relates to a method of preparing the gel and to its use for the release of said (active) ingredients onto or via the skin or the mucous membrane of a person.

Hydrogel films and their use for the administration of cosmetic ingredients onto the skin or mucosa of a person are known.

DE 3827561 C1 discloses a flexible, hydrophilic, water-swellable gel film. This gel film has a water content of 0.1 to 70% by weight and is characterized by a high water absorption capacity and good mechanical stability. The film comprises one cation-active and one anion-active polymer, and its preparation takes place using a so-called volatile additive in order to suppress the incompatibility reaction of said polymers during the dissolution operation. This gel film can be used both in cosmetics in the form of masks or films for treating the skin, and also as a pharmaceutical preparation for the transdermal administration of active ingredient.

DE 695 07 818 T2 relates to topical skincare compositions, in particular aqueous topical skincare compositions, which comprise a hydrogel thickener and a water-insoluble silicone conditioning agent. The aim here is the provision of skincare compositions which remain translucent, have an improved skin feel and exhibit reduced stickiness. The preparation of the disclosed compositions takes place by combining the constituents in accordance with conventional methods in the prior art at room temperature.

DE 699 18 418 T2 describes a haircare composition in the form of a thickened liquid based on water which has a shear gel phase which forms a reversible gel. The shear gel here is formed from a large number of separate gel particles. For the preparation, a suitable polymer is subjected to shearing during the gel formation. For the formation of the shear gel, a temperature of less than/equal to 25° C. is required.

DE 10 2004 038 285 A1 relates to drug delivery systems based on polymer matrices which comprise one or more essential oils. The polymer matrix can be composed of a synthetic polymer or a gel matrix based on agar agar/polyacrylic acid and comprise one or more permeation-controlling membranes.

It was therefore an object of the present invention to provide a cosmetic and/or pharmaceutical preparation in the form of a gel or gel film for the administration of cosmetic or pharmaceutical (active) ingredients onto or via the skin of a person, in particular for the administration of cosmetic ingredients onto the skin or mucosa of a person. This gel should therefore be suitable in particular for the cosmetic application, i.e. be highly water-containing and as skin-friendly as possible.

Furthermore, it should also be able to be easily produced without the auxiliaries and additives described in the prior art and it should also be able to be realized without the use of so-called reinforcing means, such as e.g. support layers, nonwoven inserts, chemical crosslinkers and the like, and nevertheless have adequate elasticity and minimum tear strength.

This object is achieved by the cosmetic and/or pharmaceutical preparation according to the invention in the form of a hydrogel or aquagel which is suitable for releasing pharmaceutical or cosmetic (active) ingredients onto or via the skin or the mucous membrane of a person.

The water-containing gel is preferably in the form of a flat and flexible product, in particular in the form of a so-called gel film. This product or this film preferably has a minimum layer thickness of 2 mm. The water content of the preparation is preferably more than 90% by weight, based on the finished preparation. The preparation or the hydrogel comprises at least water, one or more gel formers and one or more (active) ingredients. Moreover, the hydrogel according to the invention can comprise the further auxiliaries or additives known in the prior art, such as, for example, tackifiers for better adhesion of the gel film to the skin, preservatives and disinfectants, pH regulators, antioxidants, emulsion stabilizers, fillers, crosslinking agents, solubility promoters and humectants etc.; cf. e.g. DE 3827561 C1, in particular pages 3 to 5. Preferably, and in order to ensure the best possible skin compatibility, the number and amount of these auxiliaries and additives is restricted to a minimum. In particular, the preparation according to the invention or the hydrogel film according to the invention can be produced and used without use of the aforementioned reinforcing means; nevertheless, it has the elasticity and minimum tear strength required for the pharmaceutical or cosmetic applications.

Furthermore, compared to the products of the prior art, the gel preparation according to the invention exhibits improved optical properties e.g. with regard to the translucency and transparency. These improved optical properties are of significance in particular for the cosmetic applications of the preparation since here, besides the effectiveness of the preparation, importance is also placed on their appearance.

The preferred embodiment of the invention is in the form of the cosmetic preparation and here in particular in the use of the hydrogel or gel film according to the invention as face mask.

Even in the case of the cosmetic use, the heavily water-containing gel is preferably in the form of a flat product (e.g. as film). Besides water, preferably more than 90% by weight, the hydrogel comprises at least one cosmetic ingredient and at least one gel former. Suitable cosmetic ingredients are described, for example, in INCI (International Nomenclature of Cosmetic Ingredients). On account of the high water content of the hydrogel according to the invention, hydrophilic ingredients are particularly easy to process. The fraction of cosmetic ingredients is preferably 0.1 to 10% by weight. Preferred cosmetic ingredients are, for example, vegetable oils, synthetic oils, vitamins and vitamin derivatives, botanic extracts and active ingredients, moisturizers, skincare-substances, skin-calming and skin-smoothing substances, peeling products, antioxidants, emollients, film formers, solubility promoters (solubilizers), ionogenic and nonionogenic thickeners, penetration promoters, conditioners, sun protection filters, omega-3 and omega-6 fatty acids, amino acids, peptides, surfactants, antiinflammatories, antimicrobial active ingredients and their combinations. Dexpanthenol is particularly preferred.

Gel formers which can be used are in principle all gel formers known for the formation of hydrogels, e.g. E 400—alginic acid, E 401—sodium alginate, E 402—potassium alginate, E 403—ammonium alginate, E 404—calcium alginate, E 405—propylene glycol alginate, E 406—agar agar, E 407—carrageen, E 407a—Eucheuma algae, E 410—carob seed flour, E 412—guar seed flour, E 413—tragacanth, E 414—gum arabic, E 415—xanthan, E 416—karaya gum, E 417—tara seed flour, E 418—gellan, E 420—sorbitol, E 421—mannitol, E 422—glycerol or E 425—konjac root (E XXX=E number of the food additives approved in the European Union). Particularly preferred gel formers are (hydrophilic) polysaccharide derivatives or mixtures comprising these, also with other substances such as, for example, the various Kelcogel® grades (gellan gum) from CPKelco, Atlanta, USA, or guar gum grades or derivatives of guar seed flour such as guar hydroxypropyltrimonium chloride (commercially available from Rhodia, France, from their series with the trade mark Jaguar). The particularly preferred Kelcogel grades are mixtures comprising gellan gum, which consists of acylated linear chains with glucose, rhamnose and glucuronic acid units. In a particularly preferred variant, the Kelcogel® is mixed with xanthan gum, a high molecular weight polysaccharide of (1,4)-linked β-D-glucose with a trisaccharide side chain on each glucose unit at position 3, consisting of a glucuronic acid unit between 2 mannoses, here particularly preferably the Keltrol® grades (e.g. Keltrol® CG from CPKelco), and also konjac mannan, also known as glucomannan, a polysaccharide of β-linked D-glucose and D-mannose. The finished hydrogel product preferably comprises 0.05 to 8.0% by weight of one or more gel formers.

The above-described auxiliaries and additives used in the prior art for water-containing gels may also be present in the cosmetic preparation according to the invention. Preferably, the hydrogel according to the invention can comprise mono- or polyhydric alcohols, e.g. glycerol, as solubility promoter and moisturizer, generally to 0.5 to 20% by weight. Furthermore, metal salts can be added as crosslinking agents, preferably calcium salts such as calcium chloride, and further ionic polymers to the hydrogel according to the invention during preparation. The metal salts are present therein preferably to 0.01 to 1.0% by weight and the ionic polymers preferably to 0.1 to 5% by weight. As already explained, in the case of the cosmetic preparation, the number and the amount of auxiliaries and additives used is kept as low as possible in particular for reasons of better skin compatibility. The desired flexibility and minimum tear strength of the hydrogel product can generally also be achieved without these additives. This is true in particular for the flat preparations which exceed the preferred minimum thickness of 2 mm.

All % by weight data refer to the finished preparation or to the finished hydrogel.

The water-containing gel according to the invention or the flat product produced therefrom can have a back layer that is impermeable for the (active) ingredient and water. Furthermore, the product according to the invention can have means which bring about secure adhesion of the product to the skin or mucous membrane. This can be done, for example, by admixing the gel with so-called tackifiers, as described, or it has an adhesive layer permeable for the (active) ingredient on the side which comes into contact with the skin. Such arrangements are known to the person skilled in the art of transdermal therapeutic systems (TTS). In the latter case, the product according to the invention has, besides the optional back layer, also in most cases a protective layer or protective film on the adhesive side. Materials for the back layer and protective layer are preferably polymer films as are described, for example, in DE 3827561 C1 on page 5, lines 9 to 33, and the figures mentioned therein.

Additionally, the gel product according to the invention can have further layers which can serve as reservoir for the (active) ingredient and/or exert an additional support function. Such arrangements are known to the person skilled in the art in the field of TTS and are described, for example, in DE 3827561 C1 on page 4, lines 56 to 68.

Reference is made expressly to all of the literature passages cited in the description. These serve as references and form part of the present description.

The present invention also covers a method of preparing the hydrogel according to, the invention or a corresponding product which ensures in particular obtaining a hydrogel with the described advantageous properties.

According to the invention, the water and optionally present solubility promoters and/or humectants and/or further auxiliaries or additives are mixed and heated to a temperature between 79 and 95° C., preferably 85 to 92° C. With stirring, the gel-forming substance or a mixture of gel-forming substances is added. Finally, the pharmaceutical active ingredients or cosmetic ingredients and—if required—the ionic compounds which induce gel formation (e.g. crosslinkers in the form of e.g. metal salts and ionic polymers such as, for example, anionic polyacrylic acid derivatives) are added to the homogeneous mass and homogenized. The liquid which is formed in the process is poured out onto a support layer (e.g. back layer) and/or into a suitable packaging (e.g. deep-drawing blister). The temperature during pouring is, like that of the preceding steps, likewise between 79 and 95° C., preferably between 80 and 88° C. Upon subsequent cooling, the aqueous liquid is converted into the homogeneous hydrogel according to the invention. Here, a cooling rate of 2 to 5° C./min is particularly preferred for developing the aforementioned product properties.

The liquid is preferably poured out into a deep-drawing blister, i.e. into a packaging produced by thermoforming a defined cavity in a plastic film, in which, upon cooling, the hydrogel is formed.

In order to obtain the hydrogel according to the invention with its advantageous properties, observing the temperature ranges described above during the individual preparation steps is decisive. This is true in particular for the filling or pouring of the initially obtained aqueous, homogeneous liquid. During this step, the liquid must be hot, i.e. have a temperature between 79 and 95° C., preferably between 80 and 90° C. and particularly preferably between 80 and 88° C.

The hydrogel according to the invention is suitable for the dermal, intradermal or transdermal release of pharmaceutical active ingredients or cosmetic ingredients in people. In particular, the gel has the property of releasing its (active) ingredients onto or via the skin over a long period and in a constant amount per time unit.

Preferably, the use of the hydrogel is in cosmetics and here particularly preferably in the form of a flat and flexible product, e.g. as face mask.

The examples below serve to illustrate the invention in more detail without limiting it to these.

### EXAMPLE 1

### Preparation of a Hydrogel According to the Invention

| | | | 500 g |
|---|---|---|---|
| | | [% by | batch |
| Phase | Formulation 1 | wt.] | [g] |
| 1. | Water | 92.56 | 462.80 |
| 2. | Glycerol 86% | 4.00 | 20.00 |
| | Kelcogel F | 0.60 | 3.00 |
| | Kelcogel LT 100 | 0.38 | 1.90 |
| | Jaguar | 0.01 | 0.05 |
| 3. | Dexpanthenol 75W | 2.00 | 10.00 |
| | Polysol AC | 0.40 | 2.00 |
| | Calcium chloride × 2 H₂O | 0.05 | 0.25 |

### Preparation:

Phase 1: Heat water to 90° C.
Phase 2: Mix glycerol, Kelcogel F, Kelcogel Lt 100 and Jaguar and slowly add to the initial charge with stirring, homogenize
Add phase 3 to the initial charge; homogenize
Pour out the mass and allow to reach room temperature over 20 to 30 min.

Water (1) is heated to 90° C. With stirring, a mixture of the gel-forming substances (2) is slowly added. Finally, the cosmetic active ingredients, preservatives and the ionic compounds which induce gel formation (3) are added to the homogeneous mass, which is homogenized with stirring. The resulting liquid is then poured onto a support layer or into a deep-drawing mold. During the subsequent cooling, the liquid is converted to a homogeneous gel.

In one preferred embodiment, the water-containing gel is processed at a temperature above 79° C. and below 95° C., pouring particularly preferably takes place between 80 and 88° C.

### EXAMPLE 2

A further example of a hydrogel according to the invention is given below. The preparation takes place analogously to example 1.

| INCI name | % by wt. | Category |
|---|---|---|
| Aqua | 93.520 | A |
| Glycerol | 3.440 | E |
| Panthenol | 1.500 | E |
| Gellan Gum | 0.980 | F |
| Phenoxyethanol | 0.380 | F |
| Calcium Chloride | 0.050 | G |
| Dehydroacetic Acid | 0.035 | G |
| Benzoic Acid | 0.035 | G |
| Lactic acid | 0.035 | G |
| Sorbic Acid | 0.015 | G |
| Guar Hydroxypropyltrimonium | 0.010 | G |
| Chloride | | |
| | 100.000 | |

### EXAMPLE 3

Preparation of a hydrogel with anti-aging formulation:

| | | |
|---|---|---|
| 1 | Water | 92.5 |
| 2 | Glycerol | 4 |
| | Kelcogel F | 0.1 |
| | Keltrol CG | 0.7 |
| | Konjac mannan | 0.3 |
| 3 | Dexpanthenol | 0.5 |
| | Vitamin C | 1.0 |
| | Euxyl PE 9010 | 0.7 |
| | Calcium chloride × 2 H₂O | 0.2 |

### Preparation:

Phase 1: Heat water to 90° C.
Phase 2: Mix glycerol, Kelcogel F, Keltrol CG and konjac mannan and slowly add to the initial charge with stirring, homogenize
Add phase 3 to the initial charge, homogenize,
Pour out the mass and allow to reach room temperature over 20 to 30 min.

### EXAMPLE 4

Preparation of a hydrogel with oil components

| | | |
|---|---|---|
| 1 | Water | 91.85 |
| 2 | Glycerol | 4 |
| | Kelcogel F | 0.1 |
| | Keltrol CG | 0.7 |
| | Konjac mannan | 0.3 |
| 3 | Dexpanthenol | 1.5 |
| | Vitamin A palmitate | 0.05 |
| | Vitamin E acetate | 0.1 |
| | Rose oil | 1.0 |
| | Cremophor RH-60 | 0.5 |
| | Euxyl PE 9010 | 0.5 |
| | Calcium chloride × 2 H₂O | 0.2 |
| 4 | Perfume | 0.2 |

Phase 1: Heat water to 90° C.
Phase 2: Mix glycerol, Kelcogel F, Keltrol CG and konjac mannan and slowly add to the initial charge with stirring, homogenize
Add phase 3 to the initial charge, homogenize
Phase 4: Add fragrance to the mass and homogenize
Pour out the mass and allow to reach room temperature over 20 to 30 min.

1. A hydrogel comprising water, at least one gel former comprising gellan gum which consists of acylated linear chains with glucose, rhamnose and glucuronic acid units, at least one pharmaceutical active ingredient or cosmetic ingredient and, optionally, further auxiliaries and additives obtainable by
a) mixing water and optionally included auxiliaries and additives which are present to form a mixture,
b) adding the gel former to the mixture formed in step a) and homogenizing the resulting mixture,
c) adding the pharmaceutical active ingredient or cosmetic ingredient and, optionally, ionic compounds which induce gel formation, to the homogenized mixture formed in step b) and homogenizing the resulting mixture,
d) pouring out the homogenized mixture from step c) onto a support and
e) cooling the poured mixture from step d) to form the hydrogel,
where steps a) to d) are carried out at a temperature between 79 and 95° C.

2. The hydrogel as claimed in claim 1, wherein said hydrogel has more than 90% by weight of water.

3. The hydrogel as claimed in claim 1 wherein the gel former is a polysaccharide derivative or a mixture of polysaccharide derivatives.

4. The hydrogel as claimed in claim 1, wherein the gel former is a mixture of gellan gum, xanthan gum and konjac mannan.

5. The hydrogel as claimed in claim 1, wherein the hydrogel comprises 0.05 to 8.0% by weight of gel former.

6. The hydrogel as claimed in claim 1, wherein said hydrogel comprises a humectant.

7. The hydrogel as claimed in claim 1, wherein the humectant is glycerol.

8. The hydrogel as claimed in claim 1, wherein said hydrogel comprises an ionic compound which induces gel formation.

9. The hydrogel as claimed in claim 8, wherein the ionic compound is a metal salt or an ionic polymer.

10. The hydrogel as claimed in claim 9, wherein the metal salt is a calcium salt and the ionic polymer is a polyacrylic acid derivative.

11. The hydrogel as claimed in claim 1, wherein said hydrogel has at least one cosmetic ingredient.

12. The hydrogel as claimed in claim 11, wherein the cosmetic ingredient is dexpanthenol.

13. The hydrogel as claimed in claim 1, wherein said hydrogel is a flat product.

14. The hydrogel as claimed in claim 13, wherein the flat product has a minimum thickness of 2 mm.

15. A method of preparing a hydrogel as claimed in claim 1 comprising performing steps a) to e) in claim 1.

16. The method as claimed in claim 15, wherein step d) is carried out at a temperature between 80 and 88° C.

17. The method as claimed in claim 15, wherein the mixture obtained in step c) is poured out into a deep-drawing blister.

18. A method of administering cosmetic ingredients onto or via the skin or the mucous membrane of a person comprising adhering a hydrogel as claimed in claim 1 onto the skin or the mucous membrane of a person.

19. The method as claimed in claim 18, wherein the hydrogel is a cosmetic face mask.
